(19) United States
(12) Patent Application Publication
(10) Pub. No.: US 2007/0232657 A1
(43) Pub. Date: Oct. 4, 2007

(54) NOVEL COMPOUNDS

(75) Inventors: Mahmood Ahmed, Singapore (SG); Neil Derek Miller, Singapore (SG); Stephen Frederick Moss, Harlow (GB); Gareth John Sanger, Harlow (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, Greenford (GB)

(21) Appl. No.: 11/676,664

(22) Filed: Feb. 20, 2007

(30) Foreign Application Priority Data
Feb. 22, 2006 (GB) 0603550.5

(51) Int. Cl. A61K 31/452; C07D 405/14

(52) U.S. Cl. 514/320; 546/196

(57) ABSTRACT

The present invention relates to a novel benzamide derivative having pharmacological activity, to processes for its preparation, to compositions containing it and to its use in the treatment of diseases treatable by 5-HT4 receptor activation.

This application claims priority from Great Britain Application No. 0603550.5 filed in the United Kingdom on Feb. 22, 2006.

The present invention relates to a novel benzamide derivative having pharmacological activity, to processes for its preparation, to compositions containing it and to its use in the treatment of diseases treatable by 5-HT4 receptor activation.

WO2005/092882 (Pfizer Japan Inc.) describes a series of 4-amino-5-halogeno-benzamide derivatives. The compounds are stated to have 5-HT4 agonist activity and are indicated to be useful in the treatment of gastrointestinal disorders. WO99/02156 and EP0445862 (Janssen Pharmaceutica N.V.) describe benzamide derivatives having gastrointestinal motility stimulating properties. WO93/16072 (SmithKline Beecham plc) describes a series of benzamide derivatives that are indicated to be useful in the treatment of gastrointestinal and CNS disorders.

5-HT4 receptor agonists facilitate cholinergic activity within the gut and increase gastrointestinal motility, leading to increased rates of gastric emptying, increased transit of material through the intestine and an increased urge to defecate (Armstrong D., Current Opinion in Pharmacology, 5(6):589-95, 2005; Kamm M. A., Acta Chirurgica, Supplement. (587):10-5, 2002; Cremonini F., Delgado-Aros S. and Talley N J., Best Practice & Research in Clinical Gastroenterology, 18(4):717-33, 2004). This action can also be illustrated in vitro, by showing an ability of a 5-HT4 receptor agonist to facilitate electrically-evoked, cholinergically-mediated contractions in rodent gastric fundus strips, a response indicative of a “prokinetic-like” response (Bassil A., Murray C., Dass N M., Muir A. and Sanger G J., Eur J Pharmacol., 524, 138-144, 2005).

Thus, 5-HT4 receptor agonists have utility in the treatment of gastrointestinal disorders, especially those associated with reduced esophageal (including the lower esophageal sphincter), gastric or intestinal motility such as gastro-esophageal reflux disease, a variety of conditions associated with gastroparesis (eg., in patients with diabetes), dyspepsia conditions, including functional dyspepsia, paralytic ileus or pseudo-obstruction, irritable bowel syndrome and conditions associated with constipation, including those which are age-, disease- or drug-induced and those which may lead to additional symptoms such as incontinence. An exemplar of such a drug is tegaserod maleate, currently marketed by Novartis for the treatment of irritable bowel syndrome.

In addition, 5-HT4 agonists such as the compound of the present invention could be useful for the treatment of cognitive impairments in neurological diseases such as Alzheimer's disease and related neurological disorders.

Alzheimer's disease is a chronic neurological disorder characterised by progressive cognitive decline, behavioural impairment and ultimately death. In the US alone, it is estimated that as many as 4.5 million people suffer from the disease, including nearly half of all people over 85 years of age. With an ageing world population, there is a clear need for effective therapies for Alzheimer's Disease.

Although Alzheimer's disease is classically associated with loss of cholinergic neurons, deficits in a number of other neurotransmitter systems have also been reported, including that of the serotonergic system. With regard to 5-HT4 receptors, there is a wealth of pre-clinical data to support the use of 5-HT4 agonists as cognitive enhancers, both from in vivo and in vitro functional studies, such as rodent cognition models and electrophysiology (e.g. Moser P. C. et al., JPET 302(2):731-41, Matsumoto M., JPET 296(3):676-82, Lucas G. et al., Biol. Psychiatry 57(8):918-25). The expression profile of the 5-HT4 receptor in the central nervous system also supports this, with high levels of expression in the hippocampus, striatum, prefrontal cortex and other limbic regions. There is also evidence that 5-HT4 receptor expression may be reduced in the course of Alzheimer's disease. This data suggests that 5HT4 agonists may be useful in the treatment of cognitive impairments associated with neurological diseases such as Alzheimer's disease.

Recent reports (e.g. Maillet M. et al., Curr. Alzheimer Res. 1(2):79-85) suggest that activation of the 5-HT4 receptor can increase the release of soluble APP-alpha, which has potent neuroprotective and memory-enhancing actions, and in addition enhance the production of neurotrophic factors such as NGF and BDNF. Thus 5-HT4 agonism could also provide a disease-modifying treatment for Alzheimer's disease.

In one embodiment of the invention there is provided 5-amino-6-bromo-N-{[1-(tetrahydro-2H-pyran-4-ylmethyl)-4-piperidinyl]methyl}-3,4-dihydro-2H-chromene-8-carboxamide and/or a pharmaceutically acceptable derivative thereof.

In another embodiment of the invention there is provided a compound of formula (A), 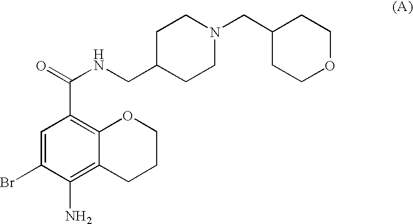
and/or a pharmaceutically acceptable derivative thereof.

In a further embodiment of the invention there is provided 5-amino-6-bromo-N-{[1-(tetrahydro-2H-pyran-4-ylmethyl)-4-piperidinyl]methyl}-3,4-dihydro-2H-chromene-8-carboxamide (A), 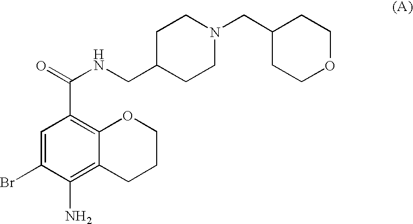
and/or a pharmaceutically acceptable derivative thereof.

When used herein “compound of the invention” means 5-amino-6-bromo-N-{[1-(tetrahydro-2H-pyran-4-yl methyl)-4-piperidinyl]methyl}-3,4-dihydro-2H-chromene-8-carboxamide and/or a pharmaceutically acceptable derivative thereof.

By pharmaceutically acceptable derivative is meant any pharmaceutically acceptable salt or solvate of 5-amino-6-bromo-N-{[1-(tetrahydro-2H-pyran-4-ylmethyl)-4-piperidinyl]methyl}-3,4-dihydro-2H-chromene-8-carboxamide, or any other compound which upon administration to the recipient is capable of providing (directly or indirectly) 5-amino-6-bromo-N-{[1-(tetrahydro-2H-pyran-4-ylmethyl)-4-piperidinyl]methyl}-3,4-dihydro-2H-chromene-8-carboxamide or an active metabolite or residue thereof. In one embodiment of the invention pharmaceutically acceptable derivative means salt or solvate. In one embodiment of the invention pharmaceutically acceptable derivative means salt. In one embodiment of the invention pharmaceutically acceptable derivative means solvate.

5-amino-6-bromo-N-{[1-(tetrahydro-2H-pyran-4-ylmethyl)-4-piperidinyl]methyl}-3,4-dihydro-2H-chromene-8-carboxamide may form acid addition salts. Such salts can be formed by reaction of the free base molecule (A) with a suitable inorganic or organic acid (such as hydrobromic, hydrochloric, sulfuric, nitric, phosphoric, succinic, maleic, mandelic, formic, acetic, propionic, fumaric, citric, tartaric, lactic, benzoic, salicylic, glutamic, aspartic, p-toluenesulfonic, benzenesulfonic, methanesulfonic, ethanesulfonic, 1,2-ethanedisulfonic, naphthalenesulfonic such as 2-naphthalenesulfonic, or hexanoic acid), optionally in a suitable solvent such as an organic solvent, to give the salt which can be isolated for example by crystallisation and filtration.

In one embodiment of the invention there is provided 5-amino-6-bromo-N-{[1-(tetrahydro-2H-pyran-4-ylmethyl)-4-piperidinyl]methyl}-3,4-dihydro-2H-chromen-8-carboxamide tosylate. In one embodiment of the invention there is provided 5-amino-6-bromo-N-{[1-(tetrahydro-2H-pyran-4-yl methyl)-4-piperidinyl]methyl}-3,4-dihydro-2H-chromene-8-carboxamide hydrochloride. In one embodiment of the invention there is provided 5-amino-6-bromo-N-{[1-(tetrahydro-2H-pyran-4-ylmethyl)-4-piperidinyl]methyl}-3,4-dihydro-2H-chromene-8-carboxamide succinate.

When the compound of the invention is allowed to crystallise or is recrystallised from organic solvents, solvent of crystallisation may be present in the crystalline product. This invention includes within its scope such solvates. Similarly, the compound of this invention may be crystallised or recrystallised from solvents containing water. In such cases water of hydration may be formed. This invention includes within its scope stoichiometric hydrates as well as compounds containing variable amounts of water that may be produced by processes such as lypohilisation. In addition, different crystallisation conditions may lead to the formation of different polymorphic forms of crystalline products. This invention includes within its scope all polymorphic forms of the compound of the invention.

Thus, the invention includes within its scope all possible stoichiometric and non-stoichiometric forms of the compound of the invention including anhydrates, hydrates, solvates and polymorphs thereof.

Since the compound of the invention is intended for use in pharmaceutical compositions, it will be understood that it is ideally used in substantially pure form, for example at least 75% pure and preferably at least 95% pure (% are on a wt/wt basis). Impure preparations of the compound of the invention may be used for preparing the more pure forms used in the pharmaceutical compositions. Although the purity of intermediate compounds of the present invention is less critical, it will be readily understood that the substantially pure form is preferred as for the compound of the invention. Whenever possible, the compound of the invention is obtained in crystalline form

The present invention also includes within its scope isotopically-labelled forms of the compound of the invention. Such compounds are identical to the compound of the invention except that one or more atoms therein are replaced by an atom having an atomic mass or mass number different from the atomic mass or mass number usually found in nature. Examples of isotopes that can be incorporated into the compound of the invention and pharmaceutically acceptable salts thereof include isotopes of hydrogen, carbon, nitrogen, oxygen and chlorine, such as 2H, 3H, 11C, 13C, 14C, 15N, 17O, 18O and 82Br.

Isotopically-labelled compounds of the present invention, for example those into which radioactive isotopes such as 3H, 14C are incorporated, are useful in drug and/or substrate tissue distribution assays. Tritiated, i.e., 3H, and carbon-14, i.e., 14C, isotopes are particularly preferred for their ease of preparation and detectability. 11C isotopes are particularly useful in PET (positron emission tomography), and are useful in brain imaging. Further substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labelled forms of the compound of the invention may be prepared by carrying out the synthetic procedures disclosed in the Schemes and/or in the Examples below, by substituting a readily available isotopically labelled reagent for a non-isotopically labelled reagent.

The present invention also provides processes for the preparation of the compound of the invention. One such process comprises reacting 5-amino-6-bromo-N-(4-piperidinylmethyl)-3,4-dihydro-2H-chromene-8-carboxamide or a salt or protected derivative thereof with tetrahydro-2H-pyran-4-carbaldehyde or a protected derivative thereof; and optionally thereafter (i) deprotecting a protected compound and/or (ii) forming a pharmaceutically acceptable derivative of the compound so formed. This process typically comprises the use of reductive conditions (such as treatment with a borohydride, for example, sodium triacetoxyborohydride) optionally in the presence of an acid catalyst, such as acetic acid, in an appropriate solvent such as 1,2-dichloroethane at a suitable temperature such as room temperature.

An alternative process for the preparation of the compound of the invention comprises reacting 5-amino-6-bromo-3,4-dihydro-2H-chromene-8-carboxylic acid or a salt or protected derivative thereof with {[1-(tetrahydro-2H-pyranylmethyl)-4-piperidinyl]methyl}amine or a salt or protected derivative thereof; and optionally thereafter (i) deprotecting a protected compound and/or (ii) forming a pharmaceutically acceptable derivative of the compound so formed. This process typically comprises the use of amide formation conditions (such as treatment with a coupling agent, for example, 1-ethyl-3(3-dimethylaminopropyl)carbodiimide hydrochloride in the presence of a base (for example 4-(N,N-dimethylamino)pyridine), in an appropriate solvent such as dichloromethane, at an appropriate temperature such as room temperature.

Examples of protecting groups and the means for their removal can be found in T. W. Greene ‘Protective Groups in Organic Synthesis’ (J. Wiley and Sons, Third Edition, 1999). Suitable amine protecting groups include sulfonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl) groups, which may be removed by hydrolysis under acidic or basic conditions (e.g. using an acid such as hydrochloric acid in dioxan or trifluoroacetic acid in dichloromethane, or using a base such as aqueous sodium hydroxide) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

5-Amino-6-bromo-3,4-dihydro-2H-chromene-8-carboxylic acid (III) and 5-amino-6-bromo-N-(4-piperidinylmethyl)-3,4-dihydro-2H-chromene-8-carboxamide (VI) may be prepared in accordance with Scheme 1. 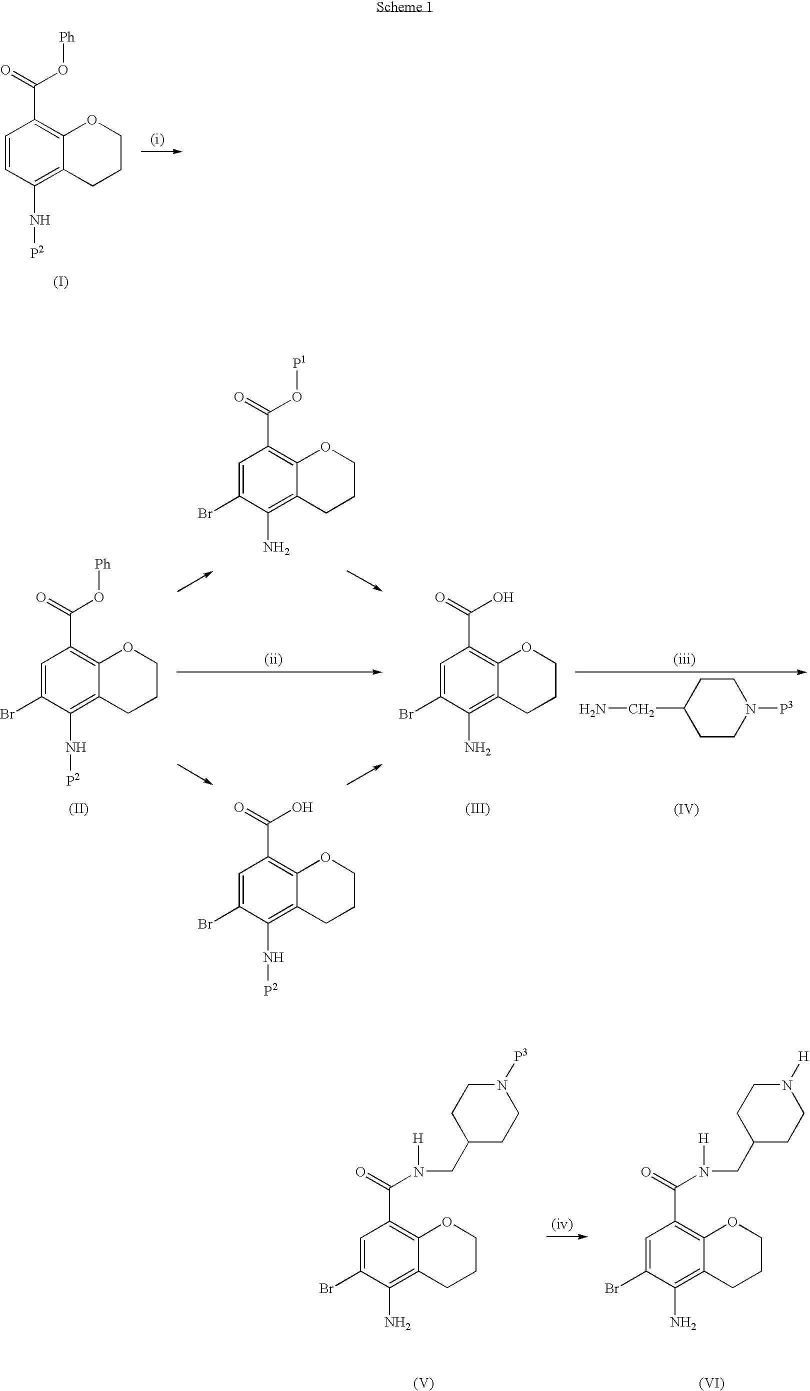
wherein P¹ represents a suitable protecting group such as an alkyl group (e.g. methyl), wherein P² represents a suitable amine protecting group such as acetyl and wherein P³ represents a suitable amine protecting group such as t-butoxycarbonyl.

Step (i) comprises a bromination reaction to provide methyl 5-(acetylamino)-6-bromo-3,4-dihydro-2H-chromene-8-carboxylate (II). A suitable brominating agent is N-bromosuccinimde. The reaction is typically performed in the presence of a suitable solvent or mixture of solvents such as 1,4-dioxan and acetic acid, at a suitable temperature, such as ambient temperature.

Step (ii) comprises a deprotection reaction to provide 5-amino-6-bromo-3,4-dihydro-2H-chromene-8-carboxylic acid (III). Where P¹ and P² cannot be removed under the same conditions, this step comprises two sequential reactions (one reaction removing P¹ and the other removing P²). However, when P¹ and P² may be removed under the same conditions (e.g. when P¹ represents methyl and P² represents acetyl), this step may consist of a single reaction. The deprotection reactions may be performed in accordance with methods known in the art, for example those described in Greene vida ante. Where P¹ represents methyl and P² represents acetyl, both protecting groups may be removed by hydrolysis under basic conditions for example using NaOH (aq)/ethanol.

Step (iii) typically comprises the use of amide formation conditions, such as treatment with a coupling agent (e.g. N-(3-dimethylaminopropyl)-N′-ethylcarbodiimide or a salt thereof, for example, hydrochloride), in an appropriate solvent such as dichloromethane.

Step (iv) comprises a deprotection reaction to provide 5-amino-6-bromo-N-(4-piperidinylmethyl)-3,4-dihydro-2H-chromene-8-carboxamide (VI) and can be performed in accordance with methods known in the art, for example those described in Greene vida ante. Where P³ represents t-butoxycarbonyl, this may be removed by hydrolysis under acidic conditions, for example using 4M HCl in 1,4-dioxan.

{[1-(Tetrahydro-2H-pyranylmethyl)-4-piperidinyl]methyl}amine may be prepared in accordance with Scheme 2. 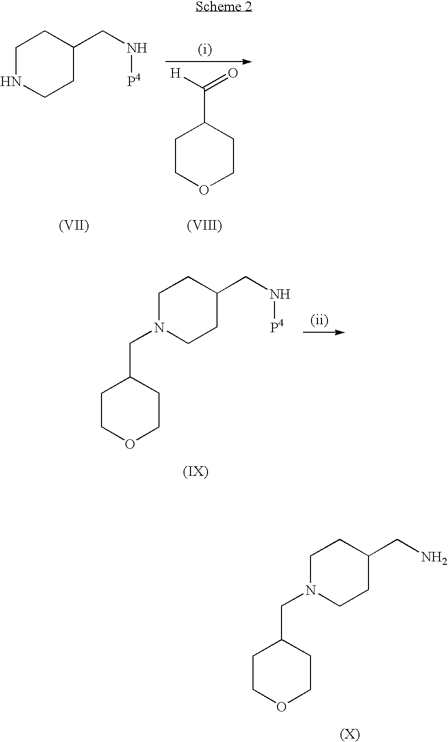
wherein P⁴ represents a suitable amine protecting group such as t-butoxycarbonyl.

Step (i) typically comprises the use of reductive conditions (such as treatment with a borohydride, for example, sodium triacetoxyborohydride), in an appropriate solvent such as 1,2-dichloroethane or dichloromethane at a suitable temperature such as ambient temperature.

Step (ii) comprises a deprotection reaction to provide {[1-(tetrahydro-2H-pyranylmethyl)-4-piperidinyl]methyl}amine (X) or a salt thereof, and can be performed in accordance with methods known in the art, for example those described in Greene vida ante. Where P⁴ represents t-butoxycarbonyl, this may be removed by hydrolysis under acidic conditions for example using 4M HCl in 1,4-dioxan.

Compounds of formula (I) may be prepared as described in US2004181064. Compounds of formula (IV), (VII) and (VIII) are either commercially available (for example from Sigma Aldrich, Epsilon Chemie and PharmaCore, respectively) or may be prepared from commercially available materials by standard methods.

Scheme 3 provides a representative process for the preparation of 5-amino-6-bromo-N-{[1-(tetrahydro-2H-pyran-4-ylmethyl)-4-piperidinyl]methyl}-3,4-dihydro-2H-chromene-8-carboxamide or a pharmaceutically acceptable derivative thereof, via 5-amino-6-bromo-3,4-dihydro-2H-chromene-8-carboxylic acid.

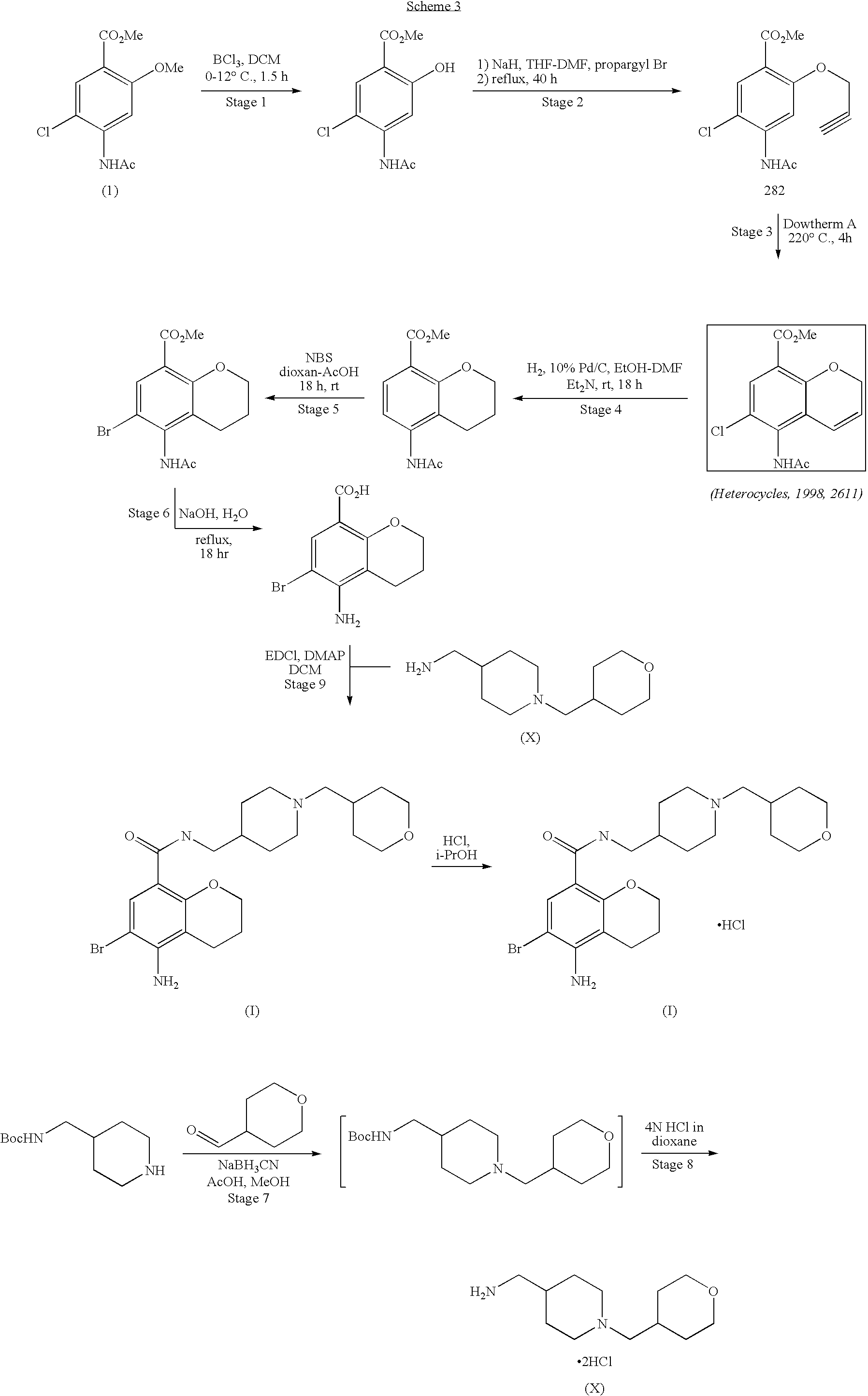

Compound (1) is commercially available (for example, from Sigma Aldrich) or may be prepared from commercially available materials such as methyl 4-(acetylamino)-2-(methyloxy)benzoate (Apin Chemicals) by standard methods.

The compound of the present invention is a partial agonist of the 5-HT4 receptor. Hence, it is believed to be of potential use in the treatment of diseases treatable by 5-HT4 agonism. Diseases treatable by 5-HT4 agonism include diseases of the central nervous system such as Alzheimer's disease and related neurological disorders, such as other dementias, cognitive disorder (especially mild cognitive impairment), generalised anxiety disorder, migraine, Parkinson's disease, multiple sclerosis, depression and schizophrenia. Diseases which may benefit from the application of a 5-HT4 receptor agonist also include functional gastrointestinal (GI) diseases such as gastroesophageal reflux disease, gastric motility disorders such as gastroparesis, non-ulcer dyspepsia, functional dyspepsia, irritable bowel syndrome, constipation, dyspepsia, esophagitis, gastroesophageal disease, nausea, emesis, inflammatory bowel disease, post-operative ileus and visceral hypersensitivity as well as pain, urinary dysfunction, urinary incontinence, overactive bladder, diabetes and apnea syndrome, (especially caused by opioid administration), and cardiovascular disorders such as cardiac failure and heart arrhythmia (hereafter ‘the disorders of the invention’).

Thus the invention also provides the compound of the invention for use as a therapeutic substance in the treatment of the disorders of the invention, in particular Alzheimer's disease and related neurological disorders, and also functional GI diseases.

The invention further provides a method of treatment of the disorders of the invention, in mammals including humans, which comprises administering to the sufferer a therapeutically effective amount of the compound of the invention.

In another aspect, the invention provides the use of the compound of the invention in the manufacture of a medicament for use in the treatment of the disorders of the invention.

It is to be understood that reference to treatment includes both treatment of established symptoms and prophylactic treatment.

When used in therapy, the compound of the invention is usually formulated in a standard pharmaceutical composition. Such a composition can be prepared using standard procedures.

Thus, the present invention further provides a pharmaceutical composition which comprises the compound of the invention or a pharmaceutically acceptable salt thereof and a pharmaceutically acceptable carrier.

The present invention further provides a pharmaceutical composition for use in the treatment of the disorders of the invention which comprises a compound of the invention and a pharmaceutically acceptable carrier.

The compound of the invention may be used in combination with other therapeutic agents.

When the compound of the invention is intended for use in the treatment of Alzheimer's disease, it may be used in combination with medicaments aimed at disease modification or at symptomatic treatment of Alzheimer's disease. Suitable examples of such other therapeutic agents may be agents known to modify cholinergic transmission such as Ml muscarinic receptor agonists or allosteric modulators, nicotinic receptor agonists or allosteric modulators, symptomatic agents such as 5-HT6 receptor antagonists or H3 receptor antagonists, also NMDA receptor antagonists (such as memantine hydrochloride) or modulators or acetylcholinesterase inhibitors (such as donepezil hydrochloride), and disease modifying agents such as β- or γ-secretase inhibitors.

When the compound of the invention is intended for use in the treatment of gastrointestinal disease, it may be used to ease the gastrointestinal side effects of other medicaments, for example, it may be used in combination with medicaments which induce symptoms treated by 5-HT4 receptor agonists. Alternatively or additionally it may be used in combination with medicaments indicated to be useful as treatments of the same or different aspects of a gastrointestinal disease. Suitable examples of therapeutic agents which evoke symptoms treated by 5-HT4 receptor agonists include those which evoke constipation, such as morphine or other opiate receptor ligands. Suitable examples of other therapeutic agents used to treat gastrointestinal disease include those known to modify gastric acid secretion, such as ranitidine or lansoprazole; gastrointestinal motility, such as almivopan, grehlin agonists or motilin agonists; or visceral pain, such as codeine; as well as the use of these compounds to aid the therapeutic use of agents designed as laxatives.

When the compound of the invention is used in combination with other therapeutic agents, the compound and agent may be administered either sequentially or simultaneously by any convenient route.

The invention thus provides, in a further aspect, a combination comprising the compound of the invention together with a further therapeutic agent or agents.

The combinations referred to above may conveniently be presented for use in the form of a pharmaceutical composition and thus pharmaceutical compositions comprising a combination as defined above together with a pharmaceutically acceptable carrier or excipient comprise a further aspect of the invention. The individual components of such combinations may be administered either sequentially or simultaneously in separate or combined pharmaceutical compositions.

When the compound of the invention is used in combination with a second therapeutic agent active the dose of each compound may differ from that when the compound is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms may be prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions may be prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.005 to 1000 mg, more suitably 0.1 to 200 mg and even more suitably 1.0 to 200 mg. In one aspect, a suitable unit dose would be 0.1-50 mg. Such unit doses may be administered more than once a day, for example two or three a day. Such therapy may extend for a number of weeks or months.

Experimental Procedures

The following Descriptions and Example illustrate the preparation of the compound of the invention. Descriptions refer to intermediate compounds.

Where indicated, chromatography was carried out on silica gel cartridges on a Flashmaster II automated chromatography system (Argonaut) and eluting with mixtures of methanol/dichloromethane or ethyl acetate/pentane.

HPLC chromatography was performed on an Agilent 1100 series HPLC machine operated in reverse phase mode using a Waters 3.9mm×150 mm Symmetry C18, 5 micron reverse phase column. A graded mixture of two solvents (labelled A and B) was used to elute samples through the column, in which solvent A was 0.1% trifluoroacetic acid in water and solvent B was 70% acetontrile in water. The gradient applied to each run was 10% B: 90% A changing over 15 minutes to 90% B: 10% A at a flow rate of 1 ml/minute. The gradient was then held at 90% B for a further 5 minutes at the same flow rate. The detection was recorded at three wavelengths (220, 254 and 270nm) and purity of components was expressed as % from Area Under Curve calculations.

¹H NMR spectra were recorded on a BrukerAVANCE 400 NMR spectrometer or a Bruker DPX250 NMR spectrometer. Chemical shifts are expressed in parts per million (ppm, δ units). Coupling constants (J) are in units of hertz (Hz). Splitting patterns describe apparent multiplicities and are designated as s (singlet), d (doublet), t (triplet), q (quartet), dd (double doublet), dt (double triplet), m (multiplet), br (broad).

LC/Mass spectra were obtained using an Agilent 1100 series HPLC system coupled with a Waters ZQ Mass Spectrometer. LC analysis was performed on a Waters Atlantis™ dC₁₈ column (50×4.6 mm, 3 μm) (mobile phase: 97% [water+0.05% HCO₂H]/3% [CH₃CN+0.05% HCO₂H] for 0.1 min, then a gradient to 3% [water+0.05% HCO₂H]/97% [CH₃CN+0.05% HCO₂H] over 3.9 min, and then held under these conditions for 0.8 min); temperature=30° C.; flow rate=3 mL/min; Mass spectra were collected using electrospray and/or APCI. In the mass spectra only one peak in the molecular ion cluster is reported. The UV detection range is from 220 to 330nm.

Reactions were typically monitored by thin-layer chromatography on 0.25 mm E. Merck silica gel plates (60F-254), visualised with UV light, 5% ethanolic phosphomolybdic acid, p-anisaldehyde solution, aqueous potassium permanganate or potassium iodide/platinum chloride solution in water.

Abbreviations
AcOH Acetic acid
DCM Dichloromethane
DCE 1,2-Dichloroethane
DMAP 4-(N,N-dimethylamino)pyridine
DMF Dimethylformamide
DMSO Dimethylsulfoxide
EDCI N-(3-dimethylaminopropyl)-N′-ethylcarbodiimide hydrochloride
EtOH Ethanol
Et₃N Triethylamine
Et₂O Diethyl ether
EtOAc Ethyl acetate
i-PrOH/IPA Isopropyl alcohol
MeOH Methanol
NBS N-bromosuccinimde
THF Tetrahydrofuran
tlc Thin layer chromatography

Description 1

### Methyl 5-(acetylamino)-6-bromo-3,4-dihydro-2H-chromene-8-carboxylate (D1)

N-Bromosuccinimide (5.9 g, 33 mmol) was added portionwise over 15 minutes to a stirred solution of methyl 5-(acetylamino)-3,4-dihydro-2H-chromene-8-carboxylate (may be prepared according to the procedure described in US2004181064) (7.9 g, 32 mmol) in 1,4-dioxan (100 ml) and glacial acetic acid (100 ml) at room temperature. No noticeable exotherm observed but solution yellowed during addition. The solution was stirred for 22 hours during which time precipitation occurred. LC/MS showed the reaction to be complete. The mixture was concentrated to a yellow solid which was redissolved in DCM (600 ml) and washed with saturated NaHCO₃ solution (400 ml), dried (MgSO₄) and concentrated to a yellow solid. This was stirred with Et₂O (200 ml) and filtered, washed with Et₂O (2×50 ml) and dried to afford the title compound (9.2 g, 28 mmol, 88%).

δH (CDCl₃, 400 MHz) 1.94-2.01 (2H, m), 2.25 (3H, s), 2.71-2.75 (2H, m), 3.87 (3H, s), 4.29 (2H, t, J=4.8 Hz), 6.99 (1H, br, s), 7.88 (1H, s). Mass Spectrum: C₁₃H₁₄BrNO₄ requires 327/329; found 328/330 (MH⁺)

Description 2

### 5-Amino-6-bromo-3,4-dihydro-2H-chromene-8-carboxylic acid (D2)

A suspension of methyl 5-(acetylamino)-6-bromo-3,4-dihydro-2H-chromene-8-carboxylate (may be prepared as described in Description 1) (9.2 g, 28 mmol) in 10% NaOH solution (200 ml) was stirred at reflux for 16 hours. The solution was cooled to room temperature and brought to pH4 with concentrated HCl (40 ml) at which point an off-white precipitate occurred. The solid was azeotroped with EtOH (2×300 ml) to remove water, filtered and the powder dried at 50° C. and identified as the title compound (D2) (7.7 g, 28 mmol, 100%) δH (DMSO-d6, 400 MHz) 1.91-1.97 (2H, m), 2.46 (2H, t, J=5.2 Hz), 4.11 (2H, t, J=5.2 Hz), 5.69 (2H, br, s), 7.66 (1H, s), 11.8 (1H, br, s).

Mass Spectrum: C₁₀H₁₀BrNO₃ requires 271/273; found 272/274 (MH⁺).

Description 3

### 1,1-Dimethylethyl 4-({[(5-amino-6-bromo-3,4-dihydro-2H-chromen-8-yl)carbonyl]amino}methyl)-1-piperidinecarboxylate (D3)

5-amino-6-bromo-3,4-dihydro-2H-chromene-8-carboxylic acid (may be prepared as described in Description 2) (1.0 g, 3.7 mmol) was dissolved in dichloromethane (80 ml). 1,1-dimethylethyl 4-(aminomethyl)-1-piperidinecarboxylate (0.78 g, 3.7 mmol; available commercially from Fluka) was dissolved in dichloromethane (40 ml) and added to the reaction mixture. 1-ethyl-3(3-dimethylaminopropyl)carbodiimide hydrochloride (1.17 g, 6.1 mmol) then 4-(N,N-dimethylamino)pyridine (72 mg, 0.59 mmol) were added. The reaction mixture was left to stir at ambient temperature for 3.5 hours. Volatiles were removed in vacuo. The residue was partitioned between DCM and water. The aqueous layer was extracted with DCM ×3. Combined organics were washed with NaHCO₃ (saturated, aqueous), then brine, and dried over MgSO₄. Volatiles were removed in vacuo to yield a yellow solid/oil The crude product was purified by chromatography using silica gel eluting with a solvent gradient of ethyl acetate/hexane (100 g column, 25%→75% EtOAc: hexane). Fractions containing the product were combined and volatiles removed in vacuo to yield a white solid (1.39 g, 2.96 mmol, 81%). δH (MeOD, 400 MHz) 1.10-1.20 (2H, m), 1.54 (9H, s), 1.70-1.90 (3H, m), 2.05-2.15 (2H, m), 2.60-2.70 (2H, m, masked by solvent peak), 2.70-2.90 (2H, m), 3.30 (2H, t, J=6 Hz), 4.00-4.15 (2H, m), 4.30-4.40 (2H, m), 5.67 (2H, br s) 7.88 (1H, s), 8.11, (1H, t).

Mass Spectrum: C₂₁H₃₀BrN₃O₄ requires 467/469; found 468/470 (MH⁺).

Description 4

### 5-Amino-6-bromo-N-(4-piperidinylmethyl)-3,4-dihydro-2H-chromene-8-carboxamide (D4)

1,1-dimethylethyl 4-({[(5-amino-6-bromo-3,4-dihydro-2H-chromen-8-yl)carbonyl]amino}methyl)-1-piperidinecarboxylate (may be prepared as described in Description 3) (1.39 g, 2.96 mmol) was dissolved in HCl (4N in dioxane, 12 ml) and water (5 ml) and stirred at room temperature for 2 hours. DCM (75 ml) was added to the reaction mixture. The reaction mixture was then basified to pH 14 with concentrated NaOH. The reaction mixture was diluted with water, and then the aqueous layer was extracted with DCM. The aqueous layer was then saturated with NaCl and decanted back into a separating funnel. The aqueous layer was then extracted a further 5 times with DCM. Combined organics were washed with brine and dried over MgSO4. Volatiles were removed in vacuo to yield a pale yellow solid (0.821 g, 2.2 mmol, 75%).

δH (MeOD, 400 MHz) 1.20-1.35 (2H, m), 1.70-1.85 (3H, m), 2.08 (2H, m), 2.55 (2H, t, J=6.8 Hz), 2.60-2.70 (2H, m), 3.05-3.15 (2H, m), 3.25-3.35 (2H, m, masked by solvent peak), 4.28 (2H, t, J=4.8 Hz), 7.88 (1H, s).

Mass Spectrum: C₁₆H₂₂BrN₃O₂ requires 367/369; found 368/370 (MH⁺)

### EXAMPLE 1

### 5-Amino-6-bromo-N-{[1-(tetrahydro-2H-pyran-4-ylmethyl)-4-piperidinyl]methyl}-3,4-dihydro-2H-chromene-8-carboxamide hydrochloride (E1)

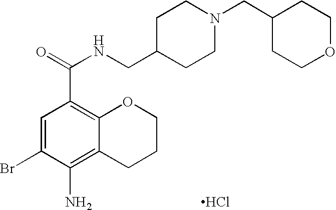

Free Base Preparation

5-Amino-6-bromo-N-(4-piperidinylmethyl)-3,4-dihydro-2H-chromene-8-carboxamide (may be prepared as described in Description 4) (100 mg, 0.27 mmol) was dissolved in DCE (5 mL). Tetrahydro-2H-pyran-4-carbaldehyde (37 mg, 0.33 mmol; available commercially from e.g. Pharmacore) was then added followed by sodium triacetoxyborohydride (144 mg, 0.68 mmol) and the reaction mixture stirred at room temperature overnight. The reaction mixture was then diluted with MeOH and loaded onto a DCM-preconditioned 10 g SCX cartridge. The column was then eluted with DCM (2 column volumes), MeOH (2 column volumes) and finally the product was eluted with 2M ammonia in MeOH (3 column volumes). Volatiles were removed in vacuo from the final fractions to yield a yellow oil. This was purified on MDAP and fractions containing product were combined and volatiles removed in vacuo to yield a yellow oil.

Hydrochloride Salt Preparation

The yellow oil was then dissolved in 2 mL of 1:1 MeOH/DCM and HCl (1 M in diethyl ether, 2 equivalents) was added. Volatiles were removed in vacuo and resultant oil was transferred to a vial using MeOH. Solvent was removed in the blow down unit and product put in vacuum oven for 30 minutes to yield the title compound as a yellow solid (E1) (100 mg, 74%).

δH (DMSO-d6, 400 MHz) 1.15-1.30 (2H, m), 1.50-1.62 (2H, m), 1.63-1.70 (2H, m), 1.72-1.85 (2H, m), 1.90-2.0 (2H, m), 2.0-2.11 (1H, m), 2.44-2.54 (3H, m, masked by solvent peak), 2.80-3.02 (4H, m), 3.15-3.24 (2H, m), 3.25-3.35 (2H, m), 3.45-3.55 (2H, m), 3.80-3.90 (2H, m), 4.15-4.25 (2H, m), 5.50 (2H, br,s), 7.73 (1H, s), 8.05 (1H, t, J=6 Hz), 9.40 (1H, br s).

Mass Spectrum: C₂₂H₃₂BrN₃O₃ requires 465/467; found 466/468 (MH⁺).

5-Amino-6-bromo-N-{[1-(tetrahydro-2H-pyran-4-ylmethyl)-4-piperidinyl]methyl}-3,4-dihydro-2H-chromene-8-carboxamide hydrochloride was also prepared according to the following procedures.

Stage 1

### Methyl 4-(acetylamino)-5-chloro-2-hydroxybenzoate

A stirred suspension of methyl 4-(acetylamino)-5-chloro-2-(methyloxy)benzoate (103 g, 0.4 mol) in dry DCM (460 ml) was cooled to 0° C. under argon. A solution of BCl₃ in DCM (1 M) (800 ml) was then cannulated directly from the reagent bottle with argon pressure by T-piece bleed control so that the rate of addition was easily controlled. The apparatus was set to control the internal temperature at 0° C. during the addition. The addition was carried out over 55 minutes. The temperature range (internally) varied 0→11° C. During the addition the apparatus was kept under argon only by the supply of BCl₃ through argon pressure. The vessel was capped with a silica drying tube. The suspension was present throughout the addition—it did not dissolve at any stage. After the complete addition of the BCl₃ solution the temperature was adjusted to 15° C. and stirred for 1.5 hours under argon. After 1.5 hours under argon the mixture was poured into water (1 L), ice (350 g) with stirring and more DCM (0.8 L) and water (0.8 L) was added to this mixture. The whole mixture was transferred to a 5 L separating funnel and vigorously shaken (solids virtually dissolved). Layers were separated and the aqueous layer extracted with DCM (2×0.8 L). The combined organic extracts were dried (MgSO₄) and combined with the same extract obtained from repeating the above-mentioned reaction using the same reactant quantities. The combined extracts from the two experiments were concentrated in vacuo to a cream solid. This solid was stirred with Et₂O (800 ml) for 10 minutes. Then hexane (800 ml) was added and the reaction continued to stir at room temperature for 0.5 hours. The solid was then filtered and washed with hexane (2×300 ml) and dried at 45° C. under vacuum for 16 hours (1 85 g, 0.759 mol, 95% th) and identified as pure product.

LCMS RT 2.44 mins (MH+)

HPLC ˜99% (RT 11.9 min) at 254 nm detection

Stage 2

### Methyl 4-(acetylamino)-5-chloro-2-(2-propyn-1-yloxy)benzoate

Sodium hydride (60% dispersion in oil, 15.1 g, 0.378 mol) added portionwise to a stirred solution of methyl 4-(acetylamino)-5-chloro-2-hydroxybenzoate (92 g) in DMF/THF (1.3 L/800 ml) under argon at 12° C. This addition took 40 minutes during which time heavy precipitation of cream solid occurred and partially dissolved towards the end of the addition. The temperature was then raised to ambient and after 1 hour propargyl bromide solution (80% w/w in toluene, 102 ml, 0.94 mol) was added over 1 minute. The solution turned dark and a slight exotherm was measured. The stirred mixture was heated at reflux under argon for 40 hours.

To this reaction mixture was added the same reaction mixture obtained from a repeat experiment using the same quantities of materials.

The combined mixture was filtered under suction to remove a small quantity of white solid. The filtrate was concentrated on a rotary evaporator (bath temperature=70° C.) to give a solid. The solid was almost dissolved in dichloromethane (4 L) and this mixture was washed with 0.5M NaOH solution (aq) (2×1 L) then water (2×1 L), dried (MgSO₄) and concentrated to a beige solid. This solid was stirred with Et₂O (550 ml) at ambient temperature for 1 hour and then filtered and washed with Et₂O (100 ml), hexane (100 ml) and the beige solid dried at 45° C under vacuum for 48 hours (155 g, 0.55 mol, 73% th) and identified as pure product.

LCMS RT 2.32 mins (MH+)

HPLC ˜99% (RT 11.1 min) at 254 nm detection

Stage 3

### Methyl 5-(acetylamino)-6-chloro-2H-chromene-8-carboxylate

Methyl 4-(acetylamino)-5-chloro-2-(2-propyn-1-yloxy)benzoate (76.4 g, 271 mmol) was added over ˜1 min to a stirred Dowtherm A (Fluka) solution (380 ml) at 215° C. (internal) under argon resulting in a temperature drop to 185° C. The temperature was re-established to 220° C. over 10 minutes and maintained at between 215-220° C. for a total time of 4 hours. The dark reaction mixture was cooled naturally to 90° C. and then poured slowly over 5 minutes into stirred hexane (3 L) at ambient temperature to give a yellow solid. After stirring for 0.5 hours it was filtered and washed with hexane (2×100 ml) then Et2O (2×100 ml), then Et2O (2×100 ml) and the solid dried at 45° C. under vacuum for 24 hours and identified as product.

LCMS RT 1.88 mins (MH+)

HPLC 97.9% (RT 8.3 min) and 2.1% (RT 11.5 min) at 254 nm detection

Stage 4

### Methyl 5-(acetylamino)-3,4-dihydro-2H-chromene-8-carboxylate

To a solution of methyl 5-(acetylamino)-6-chloro-2H-chromene-8-carboxylate (59.2 g, 210 mmol) in DMF (1.0 L) was added EtOH (1.1 L) at room temperature. Then Et₃N (29.4 ml, 210 mmol), then a slurry of 10% Pd-C (18 g) in DMF (100 ml) under argon were added. The stirred mixture was exposed to H₂ at 1 atmosphere pressure and stirred vigorously at room temperature for 18 hours. Tlc showed however that the reaction was complete after 3.5 hours. The reaction mixture was filtered through a pad of Kieselghur and washed with EtOH (2×100 ml). The filtrate was concentrated on a rotary evaporator to solid (water bath 42° C.). The solid was partitioned between dichloromethane (700 ml) and water (500 ml). The aqueous phase was re-extracted with CH₂Cl₂ (200 ml). Combined organic extracts were rewashed with water (300 ml), dired (MgSO₄), and concentrated to solid. The solid was stirred at room temperature with Et₂O (400 ml), filtered, washed with Et₂O (2×60 ml) and dried at 45° C. under vacuum for 24 hours (44 g, 176 mmol, 84% th) and identified as pure product.

LCMS RT 1.70 mins (MH+)

HPLC ˜99% (RT 7.2 min) at 254 nm detection

Stage 5

### Methyl 5-(acetylamino)-6-bromo-3,4-dihydro-2H-chromene-8-carboxylate

NBS (18.7 g, 105 mmol) was added portionwise to a stirred, slight suspension of methyl 5-(acetylamino)-3,4-dihydro-2H-chromene-8-carboxylate (25 g, 100 mmol) in dioxane (290 ml) and glacial acetic acid (290 ml) under argon at room temperature. The addition took 25 minutes and the resulting temperature rise was 22→24° C. A solution quickly formed. The dark solution was stirred at room temperature under argon for 18 hours. (LCMS showed the reaction mixture had gone to completion after 9 h; product RT 1.89 min). The reaction mixture was concentrated to oil which was redissolved in DCM (700 ml). To this stirred solution was slowly added saturated NaHCO₃ solution (700 ml) (violent fizzing). When the fizzing had subsided the mixture was transferred to a separating funnel, shaken and separated. The aqueous layer was re-extracted with DCM (200 ml) and the combined organic extracts were back-extracted with water (500 ml), dried (MgSO₄) and concentrated to brown solid. The solid was tirred with EtOAc/EtOH (1:3) (400 ml) at room temperature for 1 hour, then filtered and washed with Et₂O (2×100 ml) and dried at 45° C. under vacuum for 13 hours (24.4 g, 74.3 mmol, 74% th).

LCMS RT 1.89 mins (MH+)

HPLC ˜99% (RT 8.4 min) at 254 nm detection

Stage 6

### 5-Amino-6-bromo-3,4-dihydro-2H-chromene-8-carboxylic Acid

A suspension of methyl 5-(acetylamino)-6-bromo-3,4-dihydro-2H-chromene-8-carboxylate (23.3 g, 71 mmol) in a solution of NaOH (50 g) in water (500 ml) was stirred at reflux, open to air, for 18 hours. The reaction mixture was cooled to room temperature and then further cooled in an ice-bath. It was then stirred at ice temperature whilst concentrated HCl (aq) (˜110 ml) was slowly added to achieve a final funnel mixture at pH 5.0 (pH meter). The mixture was filtered under suction and the solid washed with water (3×70 ml) and sucked moderately dry. The solid was dried at 45° C. under vacuum for 20 hours (20.5 g, 75.3 mmol, 106% th) and identified as the product.

LCMS RT 2.05 mins (MH+)

HPLC ˜99% (RT 9.3 min) at 254 nm detection

Stage 7

### 1,1-dimethylethyl {[1-(tetrahydro-2H-pyran-4-ylmethyl)-4-piperidinyl]methyl{carbamate

To 1 equivalent of 1,1-dimethylethyl (4-piperidinylmethyl)carbamate (50 g, 233.64 mmol), 2 equivalents of sodium triacetoxyborohydride (99.06 g, 467.28 mmol) were added to 12 volumes (1 volume=75 ml) of DCM giving a yellow suspension which was cooled to 10° C. The tetrahydro-2H-pyran-4-carbaldehyde (26.63 g, 233.64 mmol) in 4 volumes of DCM was added via dropping funnel over 10 mins (slight exotherm noted ˜4° C.) whilst at 10° C. This mixture was then warmed to room temp and stirred for 2 hours. TLC and LC/MS showed reaction had gone to completion. The acetic acid (13.36 ml, 233.64 mmol) was added and this mixture was stirred for 30 mins. Extraction was carried out using 16 volumes of water (split into 4×4 volumes). The aqueous layer was adjusted to pH8-9 using 5N NaOH (approx 3 volumes), temperature raised slightly to ˜30° C. The aqueous layer was extracted 4 times with DCM/MeOH (9:1) (total volume 1.75 L). The DCM/MeOH layers were combined, dried using Na₂SO₄ and concentrated to give a yellow oil (26 g-wet with solvent by NMR analysis) which solidified on storage in the fridge (5° C)

Theoretical yield=72.89 g

The organic layer (˜1300 ml) was concentrated to ˜500 ml using a rotary evaporator and this was further extracted using 750 ml water. This aqueous layer was adjusted to pH8-9 using 5N NaOH and this was extracted using 750 ml 9:1 DCM/MeOH The organics were dried (Na₂SO₄) and concentrated to give a yellow oil (26.43 g-includes solvent on NMR) (@room temp). The organic layer was again concentrated further from ˜500 ml to ˜250 ml and further extracted using 750 ml water: the aqueous was adjusted to pH8-9 using 5M NaOH. This was extracted using 500 ml of 9:1 DCM/MeOH. Organics dried (Na₂SO₄) and concentrated to give a yellow oil (23.7 g includes solvent on NMR).

The 3 batches from the extractions were combined and held under high vacuum overnight (66.8 g).

LCMS RT 1.10 mins MH⁺313

Stage 8

### {[1-(tetrahydro-2H-pyran-4-ylmethyl)-4-piperidinyl]methyl}amine

Part A

### {[1-(tetrahydro-2H-pyran-4-ylmethyl)-4-piperidinyl]methyl}amine dihydrochloride

1,1-di methylethyl {[1-(tetrahydro-2H-pyran-4-ylmethyl)-4-piperidinyl]methyl}carbamate (66.3 g, 21.5 mmol) was dissolved in dioxane (750 ml) and cooled to 10° C. and 4M HCl was added over 20 mins. A white suspension was observed and this mixture was stirred for 1 hr. The suspension was filtered and washed with 150 ml of heptane to give a pale yellow solid which was dired in the vacuum oven overnight at 40° C. (60.34 g) The molecular ion was not detectable in the LCMS spectrum

Part B

{[1-(tetrahydro-2H-pyran-4-ylmethyl)-4-piperidinyl]methyl}amine

The SCX silica was made into slurry in MeOH and packed into a glass sinter column (130 mm diameter)- under gravity. {[1-(tetrahydro-2H-pyran-4-ylmelthyl)-4-piperidinyl]methyl}amine dihydrochloride (59.84 g, 210.7 mmol) was dissolved in 150 ml MeOH and added to the top of the SCX silica in an even band. Sand (2-3 cm) was then added to protect the surface of the silica. MeOH (5 L) was eluted through the column followed by 3 L of 2M NH₃ in MeOH. The basic fractions from the column were combined and concentrated to give a golden yellow oil (37 g) which was stored under argon in the fridge.

LCMS RT 0.30mins MH⁺213

Stage 9

### 5-Amino-6-bromo-N-{[1-(tetrahydro-2H-pyran-4-ylmethyl)-4-piperidinyl]methyl}-3,4-dihydro-2H-chromene-8-carboxamide

To a stirred solution of {[1-(tetrahydro-2H-pyran-4-ylmethyl)-4-piperidinyl]methyl}amine (10.1 g, 47.5 mmol) (see note*) in DCM (390 ml) under argon at room temperature was successively added the following reagents: 5-Amino-6-bromo-3,4-dihydro-2H-chromene-8-carboxylic acid (13.0 g, 47.8 mmol); then after 3 minutes DMAP (0.584 g, 4.8 mmol), then after 3 minutes N-(3-dimethylaminopropyl)-N′-ethylcarbodiimide hydrochloride (13.7 g, 71.5 mmol) was added over 30 seconds. The resulting mixture was stirred at room temperature under argon for 18 hours.

To the reaction mixture was added water (400 ml) and the mixture was shaken and filtered under suction through a glass sintered funnel (removing dark brown solid). The filtrate layers were separated and the aqueous layer further extracted with DCM (100 ml). The combined organics were rewashed with water (300 ml) and the latter back-extracted with DCM (100 ml). The combined organics were washed with 5% NaHCO₃ aqueous solution (500 ml), then dried (MgSO₄) and concentrated to a beige foam (16.9 g).

{[1-(tetrahydro-2H-pyran-4-ylmethyl)-4-piperidinyl]methyl}amine weighed out under argon since exposure to atmosphere leads to solidification.

LCMS RT 1.52 min (MH+for product; 94%) with impurity at RT 1.46 min (MH⁺ for by-product, 6%).

A repeat stage 9 experiment was carried out, using the same relative proportions of reactants to that described above, using 20.5 g of the stage 6 acid (total acid used in both runs=0.123 mol, on which stage 9 yield is based). The equivalent crude foam product from the second run was combined with crude product from the first run to give a combined crude product (56 g). This material was purified by silica gel chromatography using Biotage 150 system and eluting with an increasing gradient of methanol/DCM (0, 2, 4, 5, 6, 8 and 10%). Selected fractions were combined and concentrated to an oil (33.8 g) identified as the crude product free base.

HPLC 94.4% (RT 7.67min) and 5.6% (RT 7.43min) at 254 nm detection

Stage 10

### 5-Amino-6-bromo-N-{[1-(tetrahydro-2H-pyran-4-ylmethyl)-4-piperidinyl]methyl}-3,4-dihydro-2H-chromene-8-carboxamide hydrochloride

5-Amino-6-bromo-N-{[1-(tetrahydro-2H-pyran-4-ylmethyl)-4-piperidinyl]methyl}-3,4-dihydro-2H-chromene-8-carboxamide (8.04 g) was dissolved in IPA (80 ml). Concentrated HCl (1.84 ml, 1.25 eq) was then added, followed by a seed crystal of the HCl salt. The solution was then stored in the fridge for 30 minutes. The side of the flask was scratched which resulted in further crystallisation. The flask was put back into the fridge for a further 10 minutes. The resultant white crystals were then filtered off and washed with ether (x3). The product was then dried in a vacuum oven at 50° C. for 2 hours. ¹H NMR showed the product to be wet (IPA) so it was returned to a vacuum oven overnight. Additional ¹H NMR analysis showed that some IPA was still present, so the solid was dissolved in hot MeOH (20 ml), then diethyl ether was added until the solution just started to turn cloudy. The solution was then cooled in the fridge for 1 hour. White crystals were filtered off, washed with ether (x3), and dried in a vacuum oven at 50° C. for 2 hours (7.39 g, 85% th) and identified as the pure product.

HPLC ˜99% RT 7.66 min (no by-product detected) at 254 nm detection.

Biological Data

The compound of the invention may be tested for in vitro biological activity in accordance with the following assays:

Advantageously, the compound of the invention exhibits gastroprokinetic activity when tested in vitro in the rat forestomach assay described herein. The compound of the invention has also advantageously been shown to exhibit 5HT2b antagonist activity.

Yeast Functional 5-HT4a Agonist Assay

Yeast (Saccharomyces cerevisiae) cells expressing the human 5-HT4a receptor were generated by integration of an expression cassette into the ura3 chromosomal locus of yeast strain MMY23. This cassette consisted of DNA sequence encoding the human 5-HT4a receptor flanked by the yeast glyceraldehyde-3-phosphate dehydrogenase (GPD) promoter to the 5′ end of 5-HT4a and a yeast transcriptional terminator sequence to the 3′ end of 5-HT4a. MMY23 expresses a yeast/mammalian chimeric G-protein alpha subunit in which the C-terminal 5 amino acids of Gpa1 are replaced with the C-terminal 5 amino acids of human Gil (as described in Brown et al. (2000), Yeast 16:11-22). Cells were grown at 30° C. in liquid Synthetic Complete (SC) yeast media (Guthrie and Fink (1991), Methods in Enzymology, Vol.194) lacking uracil, tryptophan, adenine and leucine to late logarithmic phase (approximately 6 OD600/ml).

Agonists were prepared as 10 mM stocks in DMSO. EC50 values (the concentration required to produce 50% maximal response) were estimated using serial dilutions of between 3- and 5-fold (BiomekFX, Beckman) into DMSO. pEC50 corresponds to negative log10 of molar EC50. Agonist solutions in DMSO were transferred into black microtitre plates (96- or 384-well). 5-hydroxytryptamine was used as a positive control. Cells were suspended at a density of 0.2 OD600/ml in synthetic complete (SC) media lacking histidine, uracil, tryptophan, adenine and leucine and supplemented with 1 mM 3-aminotriazole, 0.1M sodium phosphate pH 7.0, and 10-5 M fluorescein di-β-D-glucopyranoside (FDGlu, Molecular Probes). This mixture (50 ul per well for 384-well plates, 200 ul per well for 96-well plates) was added to agonist in the assay plates, to give final assay concentration 1% DMSO. After incubation at 30° C. for 24 hours, fluorescence resulting from degradation of FDGlu to fluorescein due to exoglucanase, an endogenous yeast enzyme produced during agonist-stimulated cell growth, was determined (excitation wavelength: 485 nm; emission wavelength: 535 nm) using a Spectrofluor (Tecan) or similar microtitre plate reader. Fluorescence was plotted against compound concentration and iteratively curve fitted using a four parameter fit to generate a concentration effect value. Efficacy (Emax) was calculated from the equation:
Emax=Max[compound X]−Min[compound X]/Max[5-HT]−Min[5-HT]×100%

Where Max[compound X] and Min[compound X] are the fitted maximum and minimum respectively from the concentration effect curve for compound X, and Max[5-HT] and Min[5-HT] are the fitted maximum and minimum respectively from the concentration effect curve for 5-hydroxytryptamine. Equieffective molar ratio (EMR) values were calculated from the equation:
EMR=EC50 [compound X]/EC50 [5-HT]

Where EC50 [compound X] is the EC50 of compound X and EC50 [5-HT] is the EC50 of 5-HT.

Mammalian Functional 5-HT4a Agonist Assay

Human embryonic kidney cells stably expressing the human macrophage scavenger receptor type II (HEK-293-MSR-II cells) were established in-house (Lysko PG et al., J Pharmacol Exp Ther. 289(3):1277-85). These cells were grown in humidified conditions in Minimum Essential Medium containing 10% fetal calf serum (FCS), 1× non-essential amino acids, 2 mM L-glutamine and 0.4 mg/ml geneticin at 37° C./5% CO₂.

BacMam plasmid constructs for the production of viruses for expression in mammalian cells were generated as described (Condreay J P et al., Proc. Natl. Acad. Sci. 96:127-132, Ames R et al., Receptors Channels 10(3-4):117-24). A Kpnl-EcoRV fragment encoding the human 5-HT4a receptor cDNA (Genbank accession number Y08756) or a Kpnl-EcoRV fragment encoding the human G protein Gα16 (Genbank accession number M63904), both including an upstream Kozak sequence and start codon (GCCACCATG), were inserted separately into the BacMam shuttle vector multiple cloning site (Condreay J P et al., Proc. Natl. Acad. Sci. 96:127-132). Viruses containing these inserts were then generated using the Bac-to-Bac system (Invitrogen) according to manufacturer's instructions, and further propagated in Sf9 cells to generate high-titre virus stocks.

At 90% confluence, HEK-293-MSR-II cells were harvested in Acutase by centrifugation and resuspended in 1% dialysed FBS media. BacMam viruses encoding the 5HT4a cDNA and the Gaα16 cDNA (see above) were both added to the cells at a multiplicity of infection of 5 and 10 respectively. The cells were then plated out in 96-well clear-bottomed, black-walled plates at 35,000 cells/well (in 100 μl) and incubated overnight.

The next day 50 μl Fluo4am dye (at 6 μM in Tyrodes buffer containing 1.5 mM calcium chloride and 2.5mM probenicid) was added to each well and incubated at 37° C./5% CO₂ for one hour. Cells were then washed five times in Tyrodes buffer (composition as above), with the final wash leaving 150 μl buffer in each well. Compounds to be tested for 5-HT4 agonism were prepared in 96-well plates as half-log dilution series from 4 μM to 40 pM in Tyrodes buffer (composition as above). A Fluorimetric Imaging Plate Reader (FLIPR) was used to add the compounds to the cells (50 μl per well) and to determine peak fluorescence emitted per well over the whole assay period. These data were analysed to calculate the peak fluorescence value (minus basal), then transferred to GraphPad Prism to generate a concentration effect value and thus a pEC50 (negative log10 of molar EC50) and efficacy (intrinsic activity of compound expressed as a fraction of the maximal effect of 5-HT, with the effect of 5-HT being 1).

Rat Isolated Forestomach Assay

Longitudinal muscle strips with mucosa partially removed were,obtained from the rat gastric forestomach, and suspended under an initial “load” of 1 g for isometric recording between two platinum ring electrodes, in 5 or 10 ml tissue baths containing Krebs solution bubbled with 5% CO₂/95% O₂ and maintained at 37° C. Following an equilibration period of 30 min, during which time the tissues were flushed every 15 minutes with fresh Krebs solution, tissues were stimulated with electrical field stimulation (EFS) [50V for 10 ml bath, 25V for 5 ml bath, at 2.5 Hz 0.5 ms pulse duration, for 10 s, every 1 min]. The tissues were left stimulating for at least 30 min. After obtaining consistent responses to EFS, the test compound was added to the bathing solution, each dose left in contact for at least 10 minutes. The % potentiation of contractions to EFS were measured as the area of 4 maximum EFS contractions after addition of compound relative to the area of 4 maximum EFS contractions before addition of the lowest concentration of compound. Data are expressed as means standard error of the mean; n-values are per animal used.

Results

The compound of example El was tested in the yeast functional 5-HT4a agonist assay (n=9). The results are given below:
Mean pEC50=9.3 (−0.34 SD)
EMR=0.281
Mean efficacy=94%

The compound of example E1 was also tested in the mammalian functional 5HT4a agonist assay. It exhibited agonism at the human recombinant 5-HT4a receptor with a mean pEC50 of 8.64 and a mean efficacy of 70% (n=12).

The compound of Example E1 was tested in the rat isolated forestomach assay. The results are given below:

| | | % potentiation of contractions to EFS | | |
|---|---|---|---|---|
| Concentration of E1 | | Mean | Standard error of the mean | n |
| 1 | nM | 12.89 | 9.30 | 5 |
| 10 | nM | 50.62 | 16.85 | 5 |
| 100 | nM | 89.57 | 27.78 | 5 |
| 1 | μM | 115.46 | 32.99 | 5 |
| 10 | μM | 181.32 | 38.05 | 5 |

Subsequent testing of the compound of Example E1 in the assays described herein afforded comparable results to those provided herein.

1. A compound of formula (A), 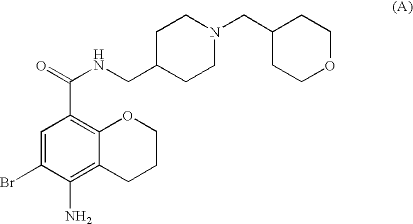
or a pharmaceutically acceptable salt thereof.

2. 5-amino-6-bromo-N-{[1-(tetrahydro-2H-pyran-4-ylmethyl)-4-piperidinyl]methyl}-3,4-dihydro-2H-chromene-8-carboxamide.

3. 5-amino-6-bromo-N-{[1-(tetrahydro-2H-pyran-4-ylmethyl)-4-piperidinyl]methyl}-3,4-dihydro-2H-chromene-8-carboxamide hydrochloride.

4. A pharmaceutical composition which comprises a compound as defined in claim 1 and a pharmaceutically acceptable carrier or excipient.

5. A pharmaceutical composition which comprises the compound as defined in claim 2 and a pharmaceutically acceptable carrier or excipient.

6. A pharmaceutical composition which comprises the compound as defined in claim 3 and a pharmaceutically acceptable carrier or excipient.

7. A method of treating Alzheimer's disease which comprises administering to a mammal in need thereof a therapeutically effective amount of a compound according to claim 1.

8. A method of treating Alzheimer's disease which comprises administering to a mammal in need thereof a therapeutically effective amount of the compound according to claim 2.

9. A method of treating irritable bowel syndrome which comprises administering to a mammal in need thereof a therapeutically effective amount of a compound according to claim 1.

10. A method of treating irritable bowel sydrome which comprises administering to a mammal in need thereof a therapeutically effective amount of the compound according to claim 2.

11. A method of treatment of diseases treatable by 5-HT4 receptor activation which comprises administering to a mammal in need thereof a therapeutically effective amount of a compound as defined in claim 1.

12. A process for the preparation of 5-amino-6-bromo-N-{[1-(tetrahydro-2H-pyran-4-ylmethyl)-4-piperidinyl]methyl}-3,4-dihydro-2H-chromene-8-carboxamide or a pharmaceutically acceptable salt thereof, which process comprises reacting 5-amino-6-bromo-N-(4-piperidinylmethyl)-3,4-dihydro-2H-chromene-8-carboxamide or a salt or protected derivative thereof with tetrahydro-2H-pyran-4-carbaldehyde or a protected derivative thereof; and optionally thereafter (i) deprotecting a protected compound and/or (ii) forming a pharmaceutically acceptable salt of the compound so formed.

13. A process for the preparation of 5-amino-6-bromo-N-{[1-(tetrahydro-2H-pyran-4-yl methyl)-4-piperidinyl]methyl}-3,4-dihydro-2H-chromene-8-carboxamide or a pharmaceutically acceptable salt thereof, which process comprises reacting 5-amino-6-bromo-3,4-dihydro-2H-chromene-8-carboxylic acid or a salt or protected derivative thereof with {[1-(tetrahydro-2H-pyranyl methyl)-4-piperidinyl]methyl}amine or a salt or protected derivative thereof; and optionally thereafter (i) deprotecting a protected compound and/or (ii) forming a pharmaceutically acceptable salt of the compound so formed.
